(19) United States
(12) Patent Application Publication
(10) Pub. No.: US 2026/0315471 A1
(43) Pub. Date: Oct. 15, 2026

(54) PAIN MODULATION USING FGFR INHIBITORS

(71) Applicant: University of Connecticut, Farmington, CT (US)

(72) Inventors: Marja Hurley, Avon, CT (US); Liping Xiao, Farmington, CT (US)

(73) Assignee: University of Connecticut, Farmington, CT (US)

(21) Appl. No.: 19/643,367

(22) Filed: Apr. 9, 2026

Related U.S. Application Data
(60) Provisional application No. 63/787,012, filed on Apr. 11, 2025.

(51) Int. Cl. A61K 31/506; A61P 25/04; C07K 16/28

(52) U.S. Cl. CPC A61K 31/506; A61P 25/04; C07K 16/2863

(57) ABSTRACT

The present disclosure describes the methods for pain management, especially sickle cell disease (SCD) pain management, using at least one fibroblast growth factor receptor (FGFR) inhibitor, a pharmaceutically acceptable salt, enantiomer, stereoisomer, solvate, polymorph or prodrug thereof, or a pharmaceutical composition including at least one fibroblast growth factor receptor (FGFR) inhibitor.

### RELATED APPLICATIONS

The instant application claims priority to U.S. Provisional Application No. 63/787,012, filed on Apr. 11, 2025. The entire contents of the foregoing application are expressly incorporated by reference herein.

### GOVERNMENT SUPPORT

This invention was made with government support under DK129431 awarded by the National Institutes of Health. The government has certain rights in the invention.

### FIELD OF THE DISCLOSURE

The disclosure is directed to pain management using at least one fibroblast growth factor receptor (FGFR) inhibitors, a pharmaceutically acceptable salt, enantiomer, stereoisomer, solvate, polymorph or prodrug thereof, especially sickle cell disease (SCD) pain modulation using an FGFR tyrosine kinase inhibitor (FGFR-TKI).

### BACKGROUND

Sickle cell disease (SCD) is the most common genetic blood disorder worldwide that impacts millions of individuals. Approximately 100,000 Americans have SCD and over 3 million people worldwide. SCD is characterized by debilitating pain that begins during infancy and continues to increase throughout life. Blockade of capillaries with sickle red blood cells induces vaso-occlusive episode (VOE), which causes severe pain and organ damage including bone damage. Treatment options for sickle cell disease pain are limited and primarily involve the use of opioids. However, long-term opioid use is associated with numerous side effects. Therefore, there is an immediate need for novel treatment options for SCD pain.

### SUMMARY

The following brief summary is not intended to include all features and aspects of the present disclosure, nor does it imply that any claim must include all features and aspects discussed in this summary.

Disclosed herein is a method for modulating pain, especially sickle cell disease (SCD) pain, in a subject, the method including providing and administrating at least one fibroblast growth factor receptor (FGFR) inhibitor, a pharmaceutically acceptable salt, enantiomer, stereoisomer, solvate, polymorph or prodrug thereof, or a pharmaceutical composition including at least one fibroblast growth factor receptor (FGFR) inhibitor to the subject as shown and described herein. In certain embodiments, administration of the FGFR inhibitor reduces mechanical, thermal, and/or deep tissue hyperalgesia through modulation of innate immune signaling in dorsal root ganglia, including decreased expression of toll-like receptor 2 (TLR2) and ionized calcium-binding adapter molecule 1 (IBA1).

In an aspect, disclosed is a composition including a fibroblast growth factor receptor (FGFR) tyrosine kinase inhibitor (TKI) for pain modulation, especially for sickle cell disease (SCD) pain modulation as shown and described herein.

In some aspects, provided herein is a method of treating pain in a subject, the method comprising administrating to the subject an effective amount of a fibroblast growth factor receptor (FGFR) inhibitor, thereby treating pain in the subject.

In some embodiments, the FGFR inhibitor is a pan-inhibitor. In some embodiments, the FGFR inhibitor is an FGFR tyrosine kinase inhibitor (FGFR-TKI). In some embodiments, the FGFR inhibitor is selected from the group consisting of BGJ398, erdafitinib, pemigatinib, futibatinib, lirafugratinib, dabogratinib, irpagratinib, dovitinib, nintedanib, lenvatinib, and ponatinib, or a pharmaceutically acceptable salt, enantiomer, stereoisomer, solvate, polymorph, or prodrug thereof. In some embodiments, the FGFR inhibitor is BGJ398 or a pharmaceutically acceptable salt, enantiomer, stereoisomer, solvate, polymorph, or prodrug thereof. In some embodiments, the FGFR inhibitor is bemarituzumab.

In some embodiments, the pain is selected from the group consisting of pain associated with a sickle cell disease, somatic pain, neuropathic pain, inflammatory pain, musculoskeletal pain, visceral pain, nociplastic pain, acute pain, and chronic pain. In some embodiments, the pain is associated with a sickle cell disease. In some embodiments, the subject exhibits one or more symptoms selected from the group consisting of mechanical hyperalgesia, deep tissue hyperalgesia, and thermal hyperalgesia. In some embodiments, administration of the FGFR inhibitor results in a reduction, elimination, or stabilization of one or more symptoms selected from the group consisting of mechanical hyperalgesia, deep tissue hyperalgesia, and thermal hyperalgesia.

In some embodiments, administration of the FGFR inhibitor decreases toll-like receptor 2 (TLR2) expression in the dorsal root ganglion (DRG) of the subject. In some embodiments, administration of the FGFR inhibitor decreases TLR2 expression in the DRG of the subject by at least 1.5-fold, at least 2-fold, at least 2.5-fold, at least 3-fold, at least 3.5-fold, at least 4-fold, at least 4.5-fold, or at least 5-fold compared to administration of a vehicle control.

In some embodiments, administration of the FGFR inhibitor decreases ionized calcium-binding adapter molecule 1 (IBA1) expression in the DRG of the subject. In some embodiments, administration of the FGFR inhibitor decreases IBA1 expression in the DRG of the subject by at least 1.5-fold, at least 2-fold, at least 2.5-fold, at least 3-fold, at least 3.5-fold, at least 4-fold, at least 4.5-fold, or at least 5-fold compared to administration of a vehicle control.

In some embodiments, administration of the FGFR inhibitor results in an improvement in the subject's score of a clinical assessment tool, as compared to a score of the clinical assessment tool prior to administration of the FGFR inhibitor. In some embodiments, the clinical assessment tool is selected from the group consisting of Numeric Rating Scale (NRS), Visual Analogue Scale (VAS), Wong-Baker FACES Pain Rating Scale, Behavioral Pain Scale, Short Form Health Survey 36, McGill Pain Questionnaire (MPQ), pressure algometry, Von Fey test, and Quantitative sensory testing (QST).

In an embodiment, the subject is a mammal such as human, cat, dog, pig, or horse. In some embodiments, the subject is a human subject.

These and other aspects of the present invention are described in more detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph depicting that BGJ398 increases the paw withdrawal threshold (PWT) for mechanical stimulation in sickle cell disease (SCD) mice. Shown in FIG. 1 is PWT (grams, g) for six months old female healthy control (Ctrl) and SCD mice treated with either vehicle (Veh) or BGJ398, 5 days/week for 5 weeks. N=6-8 mice per group. Data are presented ad mean±SEM with individual data points. *p<0.05 by two-way ANOVA.

FIG. 2 is a graph depicting that BGJ398 increases the PWT for mechanical stimulation in SCD mice. Shown in FIG. 2 is PWT (grams, g) for eight months old male healthy control (Ctrl) and SCD mice treated with either vehicle (Veh) or BGJ398, 5 days/week for 5 weeks. N=4-6 mice per group. Data are presented as mean±SEM with individual data points. *p<0.05 by two-way ANOVA.

FIG. 3 is a graph depicting that BGJ398 increases the paw withdrawal latency (PWL) for thermal stimulation in SCD mice. Shown in FIG. 3 is PWL (seconds, s) for eight months old male healthy control (Ctrl) and SCD mice treated with either vehicle (Veh) or BGJ398, 5 days/week for 5 weeks. N=4-6 mice per group. Data are presented as mean±SEM with individual data points. *p<0.05 by two-way ANOVA.

FIG. 4 is a dorsal root ganglion (DRG) gene expression heatmap showing the top five genes upregulated in the indicated pathways in DRG tissue from eight months old male control (Ctrl) and SCD mice treated with vehicle or BGJ398 (2 mg/kg body weight/day), 5 days per week for 6 weeks. Red and blue indicate upregulated and downregulated genes, respectively. n=4-6 mice per group.

FIG. 5 are representative images of immunofluorescence staining for DRG toll-like receptor 2 (TLR2) expression in six months old control (Ctrl) and SCD female treated with vehicle or BGJ398 (2 mg/kg body weight/day), five days per week for six weeks. n=3 mice per group.

FIG. 6 are representative images of immunofluorescence staining for DRG ionized calcium-binding adapter molecule 1 (IBA1) expression in six months old control (Ctrl) and SCD female mice treated with vehicle or BGJ398 (2 mg/kg body weight/day), five days per week for six weeks. n=3 mice per group.

FIGS. 7A-7C are graphs demonstrating that BGJ398 at a lower dose significantly alleviates pain-related behaviors in SCD mice. Shown are PWT (grams, g) (FIG. 7A), grip strength (grams, g) (FIG. 7B), and PWL(seconds, s) (FIG. 7C) for six months old female healthy control (Ctrl) and SCD mice treated with either vehicle (Veh) or BGJ398 (1 mg/kg body weight/day), two days per week for six weeks. n=4-6 mice per group. Data are presented as mean±SEM with individual data points. *p<0.05 by one-way ANOVA.

### DETAILED DESCRIPTION

### Overview

Inventors have surprisingly and unexpectedly discovered that FGFR inhibitors effectively modulate pain, especially pain in subjects having SCD. Generally, inventors have discovered that FGFR tyrosine kinase inhibitors (FGFR-TKIs) show effective SCD pain modulation in a humanized mouse model of sickle cell disease (SCD). Inventors have collected data of neuropathic allodynia in a humanized mouse model of sickle cell disease (SCD) compared with healthy control mice that could be significantly reduced with an FGFR tyrosine kinase inhibitor, BGJ398, also known as Infigratinib. Therefore, the FGFR tyrosine kinase inhibitors such as BGJ398 or any other FGFR tyrosine kinase inhibitors can modulate SCD pain. SCD pain is currently not well managed since opioid drugs to relieve pain are the only treatment which may lead to addiction, as there is no alternative technology to treat SCD pain, this result has created a potential translational/clinical to treat option for human SCD pain.

Accordingly, in some aspects, provided herein is a method of treating pain (e.g., pain associated with a sickle cell disease) in a subject, the method comprising administrating to the subject an effective amount of a fibroblast growth factor receptor (FGFR) inhibitor (e.g., BGJ398 or pharmaceutically acceptable salt, enantiomer, stereoisomer, solvate, polymorph, or prodrug thereof), thereby treating pain in the subject.

Without being bound by theory, the inventors believe that FGFR inhibition modulates nociceptive signaling in sickle cell disease by attenuating peripheral neuroimmune activation within dorsal root ganglia. In particular, FGFR tyrosine kinase inhibition reduces expression of TLR2 and IBA1, markers respectively associated with innate immune signaling and microglial/macrophage activation, thereby reducing hyperalgesia. This mechanism is distinct from central opioid-mediated analgesia and provides a non-opioid approach to treating pain associated with sickle cell disease.

Some unique advantages of the compositions and methods disclosed herein include:
1. Non-opioid pain management: Provides an effective alternative to opioid treatments, thereby reducing risks of addiction and adverse side effects associated with long-term opioid use in SCD patients.
2. Targeted therapeutic approach: The method utilizes FGFR inhibitor, such as BGJ398, to inhibit FGF receptor tyrosine kinase activity, offering a novel mechanism of action unlike conventional pain management strategies, such as hydroxyurea or crizanlizumab, which do not directly address pain via this pathway.
3. Dual-mode analgesic efficacy: The method demonstrated significant reduction in both mechanical and thermal hyperalgesia in preclinical SCD models, indicating a broader and more effective pain relief profile than standard opioid-based therapies.
4. Potential cost and healthcare resource savings: By lessening reliance on opioids, which often lead to additional healthcare complications and associated costs, this approach may reduce overall treatment expenses and improve patient quality of care.

Before the disclosed processes and materials are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

### Methods and Compositions

In an aspect, disclosed is a method for modulating pain, especially SCD pain, in a subject, the method includes providing and administering at least one fibroblast growth factor receptor (FGFR) inhibitor, a pharmaceutically acceptable salt, enantiomer, stereoisomer, solvate, polymorph or prodrug thereof, or a composition (also called as therapeutic composition) including at least one fibroblast growth factor receptor (FGFR) inhibitor to the subject. In an embodiment, the subject is a mammal. In an embodiment, the subject is human.

In some embodiments, the FGFR inhibitor is a pan-inhibitor (e.g., a molecule that inhibits two or more subtypes of the FGFR family, such as FGFR1, FGFR2, FGFR3, and FGFR4). In some embodiments, the FGFR inhibitor inhibits one or more FGFR family proteins selected from the group consisting of FGFR 1, FGFR 2, FGFR 3, and FGFR4.

In some embodiments, the FGFR inhibitor is an antibody or antigen binding fragment thereof (e.g., bemarituzumab).

In some embodiments, the FGFR inhibitor is a small molecule (e.g., erdafitinib, pemigatinib, futibatinib, lirafugratinib, dabogratinib, irpagratinib, dovitinib, nintedanib, lenvatinib, and ponatinib) or a pharmaceutically acceptable salt, enantiomer, stereoisomer, solvate, polymorph or prodrug thereof.

In an embodiment, the FGFR inhibitor is an FGFR tyrosine kinase inhibitor (FGFR-TKI), a pharmaceutically acceptable salt, enantiomer, stereoisomer, solvate, polymorph or prodrug thereof. In an embodiment, the FGFR-TKI is a small molecule or an antibody, a pharmaceutically acceptable salt, enantiomer, stereoisomer, solvate, polymorph or prodrug thereof. In an embodiment, the FGFR-TKI is a small molecule, a pharmaceutically acceptable salt, enantiomer, stereoisomer, solvate, polymorph or prodrug thereof. In an embodiment, the FGFR-TKI is a BGJ398, a pharmaceutically acceptable salt, enantiomer, stereoisomer, solvate, polymorph or prodrug thereof.

In an embodiment, the composition includes at least one FGFR inhibitor, a pharmaceutically acceptable salt, enantiomer, stereoisomer, solvate, polymorph or prodrug thereof. In an embodiment, the composition includes at least one FGFR-TKI, a pharmaceutically acceptable salt, enantiomer, stereoisomer, solvate, polymorph or prodrug thereof. In an embodiment, the composition includes BGJ398, a pharmaceutically acceptable salt, enantiomer, stereoisomer, solvate, polymorph or prodrug thereof. In an embodiment, the composition includes at least one carrier. In some embodiments, the FGFR inhibitor is administered as a pharmaceutical composition comprising the FGFR inhibitor or a pharmaceutically acceptable salt thereof and one or more pharmaceutically acceptable carriers, diluents, excipients, stabilizers, buffers, tonicity agents, preservatives, surfactants, solubilizers, or combinations thereof.

In some embodiments, the FGFR inhibitor is formulated for oral, parenteral, intravenous, subcutaneous, intramuscular, intraperitoneal, intrathecal, transdermal, transmucosal, intranasal, buccal, rectal, or topical administration. In some embodiments, the FGFR inhibitor is formulated for oral administration. In some embodiments, the FGFR inhibitor is formulated for parenteral administration. In some embodiments, the FGFR inhibitor is formulated for subcutaneous administration.

In some embodiments, the FGFR inhibitor is provided in the form of a tablet, capsule, powder, granule, solution, suspension, emulsion, syrup, injectable solution, injectable suspension, depot formulation, controlled-release formulation, extended-release formulation, liposomal formulation, nanoparticle formulation, implant, patch, or osmotic delivery system.

In an aspect, disclosed is a method of treating a subject, the method including: administering to the subject a therapeutic amount of a therapeutic composition, where the therapeutic composition includes at least one FGFR inhibitor, a pharmaceutically acceptable salt, enantiomer, stereoisomer, solvate, polymorph or prodrug thereof; where the therapeutic composition is operative to reduce pain associated with a disease, especially, pain associated with SCD.

In some embodiments, the pain is selected from the group consisting of pain associated with a sickle cell disease, somatic pain, neuropathic pain, inflammatory pain, musculoskeletal pain, visceral pain, nociplastic pain, acute pain, and chronic pain. In some embodiments, the pain comprises one or more (e.g., 1, 2, 3, 4, or more) types of pain selected from the group consisting of pain associated with a sickle cell disease, somatic pain, neuropathic pain, inflammatory pain, musculoskeletal pain, visceral pain, nociplastic pain, acute pain, and chronic pain.

In some embodiments, the pain is associated with a sickle cell disease (e.g., a vaso-occlusive episode).

In some embodiments, the subject exhibits one or more symptoms selected from the group consisting of mechanical hyperalgesia, deep tissue hyperalgesia, and thermal hyperalgesia. In some embodiments, administration of the FGFR inhibitor results in a reduction, elimination, or stabilization of one or more symptoms selected from the group consisting of mechanical hyperalgesia, deep tissue hyperalgesia, and thermal hyperalgesia. In some embodiments, administration of the FGFR inhibitor results in a reduction of one or more symptoms selected from the group consisting of mechanical hyperalgesia, deep tissue hyperalgesia, and thermal hyperalgesia by at least 1.5 fold, at least 2 fold, at least 3 fold, at least 4 fold, at least 5 fold, at least 6 fold, at least 7 fold, at least 8 fold, at least 9 fold, at least 10 fold, at least 50 fold, at least 100 fold, at least 500 fold, at least 1000 fold, at least 5000 fold, or at least 10,000 fold compared to administration of a vehicle control or compared to the one or more symptoms in the subject prior to administration of the FGFR inhibitor. In some embodiments, the FGFR inhibitor is an FGFR-TKI. In some embodiments, the FGFR inhibitor is BGJ398 or pharmaceutically acceptable salt, enantiomer, stereoisomer, solvate, polymorph, or prodrug thereof. Any suitable system can be used to assess the one or more symptoms in the subject (e.g., Quantitative sensory testing (QST), von Frey testing, a grip strength meter, etc.)

In some embodiments, administration of the FGFR inhibitor decreases toll-like receptor 2 (TLR2) expression in the dorsal root ganglion (DRG) of the subject. In some embodiments, administration of the FGFR inhibitor decreases TLR2 expression in the DRG of the subject by at least 1.5 fold, at least 2 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 50 fold, at least 100 fold, at least 500 fold, at least 1000 fold, at least 5000 fold, or at least 10,000 fold compared to administration of a vehicle control or compared to prior to administration of the FGFR inhibitor. In some embodiments, the FGFR inhibitor is an FGFR-TKI. In some embodiments, the FGFR inhibitor is BGJ398 or pharmaceutically acceptable salt, enantiomer, stereoisomer, solvate, polymorph, or prodrug thereof. Suitable techniques for measuring the expression of TLR2 in vivo (e.g., measurement of radiotracers using positron emission tomography (PET)) or in vitro (e.g., ELISA, western blotting, mass spectrometry, immunofluorescence, flow cytometry, etc.) will be clear to the skilled person.

In some embodiments, administration of the FGFR inhibitor decreases ionized calcium-binding adapter molecule 1 (IBA1) expression in the DRG of the subject. In some embodiments, administration of the FGFR inhibitor decreases IBA1 expression in the DRG of the subject by at least 1.5 fold, at least 2 fold, at least 3 fold, at least 3.5 fold, at least 4 fold, at least 4.5 fold, at least 5 fold, at least 6 fold, at least 7 fold, at least 8 fold, at least 9 fold, at least 10 fold, at least 50 fold, at least 100 fold, at least 500 fold, at least 1000 fold, at least 5000 fold, or at least 10,000 fold compared to administration of a vehicle control or compared to prior to administration of the FGFR inhibitor. In some embodiments, the FGFR inhibitor is an FGFR-TKI. In some embodiments, the FGFR inhibitor is BGJ398 or pharmaceutically acceptable salt, enantiomer, stereoisomer, solvate, polymorph, or prodrug thereof. Suitable techniques for measuring the expression of IBA1 in vivo (e.g., measurement of radiotracers using positron emission tomography (PET)) or in vitro (e.g., ELISA, western blotting, mass spectrometry, immunofluorescence, flow cytometry, etc.) will be clear to the skilled person.

Diagnosis and characterization of pain are guided by methods known in the art. In some embodiments, the diagnosis and characterization of pain include, but are not limited to, a review of a subject's medical history, a review of a subject's symptoms, or one or more clinical assessments. In some embodiments, administration of the FGFR inhibitor results in an improvement in the subject's score of a clinical assessment tool, as compared to a score of the clinical assessment tool prior to administration of the FGFR inhibitor. In some embodiments, the clinical assessment tool is selected from the group consisting of Numeric Rating Scale (NRS), Visual Analogue Scale (VAS), Wong-Baker FACES Pain Rating Scale, Behavioral Pain Scale, Short Form Health Survey 36, McGill Pain Questionnaire (MPQ), pressure algometry, Von Fey test, and Quantitative sensory testing (QST).

In some embodiments, the FGFR inhibitor is administered with one or more agents or therapies. For example, the FGFR inhibitor may be administered to a subject in combination with one or more sickle cell disease medications including, but not limited to, crizanlizumab, glutamine, hydroxyurea, voxeletor, rivipansel, arginine, acetaminophen, a nonsteroidal anti-inflammatory drug (NSAID), ketamine, lidocaine, gabapentin, a SNRIs (SNRI), a tricyclic antidepressant, and buprenorphine.

In some embodiments, the FGFR inhibitor is administered once daily, twice daily, once every other day, two times per week, three times per week, five times per week, once weekly, or according to another clinically appropriate schedule. In some embodiments, the FGFR inhibitor is administered once daily. In some embodiments, the FGFR inhibitor is administered five days per week. In some embodiments, the FGFR inhibitor is administered two times per week.

In some embodiments, the FGFR inhibitor is administered for at least about 3 days, at least about 5 days, at least about 1 week, at least about 2 weeks, at least about 3 weeks, at least about 4 weeks, at least about 5 weeks, at least about 6 weeks, at least about 8 weeks, at least about 12 weeks, or longer. In some embodiments, the FGFR inhibitor is administered chronically. In some embodiments, the FGFR inhibitor is administered during or following onset of a vaso-occlusive episode. In some embodiments, the FGFR inhibitor is administered between vaso-occlusive episodes to reduce, prevent, or lessen pain, hyperalgesia, or recurrence of pain symptoms.

In some embodiments, the FGFR inhibitor is administered at a dose of about 0.01 mg/kg to about 100 mg/kg, about 0.05 mg/kg to about 50 mg/kg, about 0.1 mg/kg to about 25 mg/kg, about 0.5 mg/kg to about 10 mg/kg, or about 1 mg/kg to about 5 mg/kg. In some embodiments, the FGFR inhibitor is administered at a dose of about 1 mg/kg. In some embodiments, the FGFR inhibitor is administered at a dose of about 2 mg/kg. In some embodiments, the FGFR inhibitor is BGJ398 and is administered at about 1 mg/kg to about 5 mg/kg. In some embodiments, the FGFR inhibitor is BGJ398 and is administered at about 1 mg/kg to about 2 mg/kg.

In some embodiments, the effective amount is sufficient to reduce one or more pain-associated phenotypes, including mechanical hyperalgesia, thermal hyperalgesia, or deep tissue hyperalgesia, as compared to a pretreatment baseline or a control. In some embodiments, the effective amount is sufficient to increase paw withdrawal threshold, increase paw withdrawal latency, improve grip strength, reduce a pain score, reduce pain frequency, reduce pain duration, reduce analgesic rescue medication use, or any combination thereof.

In some embodiments, administration comprises an initial dose followed by one or more maintenance doses. In some embodiments, the dose is escalated, reduced, interrupted, or discontinued based on tolerability, adverse events, subject weight, age, route of administration, clinical response, pain severity, renal function, hepatic function, concomitant medications, or combinations thereof.

In some embodiments, the FGFR inhibitor is administered as monotherapy. In some embodiments, the FGFR inhibitor is administered in combination with one or more additional therapies for sickle cell disease or pain management. In some embodiments, the FGFR inhibitor is administered simultaneously with the one or more additional therapies. In some embodiments, the FGFR inhibitor is administered before or after the one or more additional therapies.

In some embodiments, administration of the FGFR inhibitor reduces mechanical hyperalgesia, thermal hyperalgesia, and/or deep tissue hyperalgesia in a subject having sickle cell disease.

As used herein, the terms “treatment” and “treating” can include reversing, alleviating, inhibiting the progression of, preventing or reducing the likelihood of pain. Accordingly, the disclosed compositions can be administered prophylactically to prevent or reduce the incidence or recurrence of pain. In an embodiment, the subject is a mammal (e.g. a human, horse, or dog, or cat). “Pain modulation or management” means alleviation of pain in whole or in part. In an embodiment, the disclosed methods and compositions reduce the pain by at least about 20% (e.g., at least about 25%, 30%, 35%, 40%, 45%, 50%, 55%, 60%, 65%, 70%, 75%, 80%, 85%, 90%, or 95%). In an embodiment, the disclosed methods and compositions reduce the pain by more than about 90% (e.g. at least about 95%, 100%, 85%, 80%, 75%, 70%, 65%, 60%, 55%, 50%, 45%, 40%, 35%, 30%, 25%).

Generally, the methods and compositions described herein can be administered as monotherapy or as a part of combination therapy. In an embodiment, the methods described herein are administered as monotherapy. In an embodiment, the methods described herein are administered as a part of combination therapy.

### Kits

Aspects of the disclosure contemplate kits including at least one FGFR inhibitor of the present disclosure for implementing related methods of the disclosure. In an aspect, the present disclosure provides a kit for modulating pain in a subject having sickle cell disease. In some aspect, the kit can comprise instructions for carrying out the methods for pain modulation (management). Instructions included in kits can be affixed to packaging material or included as a package insert. While the instructions are typically written or printed materials, they are not limited to such. Any medium capable of storing such instructions and communicating them to an end user is contemplated by this disclosure. Such media include but are not limited to, electronic storage media (e.g., magnetic discs, tapes, cartridges, chips), optical media (e.g., CD ROM), and the like. As used herein, the term “instructions” can include the address of an internet site that provides the instructions. In an embodiment, the kit includes at least one FGFR inhibitor and/or a composition including at least one FGFR inhibitor, a pharmaceutically acceptable salt, enantiomer, stereoisomer, solvate, polymorph or prodrug thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

### Definitions

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4. 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human or a non-human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. Treatment means any way the symptoms of a pathological condition, disorder, or disease are ameliorated or otherwise beneficially altered. Preferably, the subject in need of such treatment is a mammal, preferably a human. Treatment also means providing an active compound to a patient in an amount sufficient to measurably reduce any pain symptom. These terms also encompass therapy and cure. In certain embodiments treatment of the pain may be commenced before the patient presents symptoms of the disease.

As used herein, the term “effective amount” or “therapeutically effective amount” or “prophylactically effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose. A therapeutically effective amount of the composition often is determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. As used herein, the term “prophylaxis” refers to preventing or reducing the progression of a disorder, either to a statistically significant degree or to a degree detectable by a person of ordinary skill in the art.

As used herein, the term “dose” or “dosage” denotes any form of an active ingredient formulation or composition, including cells, that contains an amount sufficient to initiate or produce a therapeutic effect with at least one or more administrations. “Formulation” and “composition” are used interchangeably herein. As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is in the prenatal period, newborn to one year of age, or about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments. The terms “subject” and “patient” are used interchangeably herein. The term “subject” also refers to an organism, tissue, cell, or collection of cells from a subject.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

“Pharmaceutical compositions” means compositions comprising at least one active agent, such as a compound or salt of FGFR inhibitor disclosed herein, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A significant change is any detectable change that is statistically significant in a standard parametric test of statistical significance such as Student's t-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### EXAMPLES

The present disclosure is illustrated and further described in more detail with reference to the following non-limiting results and examples.

### Example 1

### Methods

Mice: Control and SCD mice were treated with either vehicle (HCL 3.5 mM, DMSO 5%) or FGF Receptor Signaling Inhibitor BGJ398 2mg/kg/BW/day sq for 6 weeks.

Mechanical hyperalgesia: At 5 weeks after treatment with Vehicle or BGJ398 has commenced, the “up-down” von Frey method was used to determine the mechanical force required to elicit a paw withdrawal response (paw withdrawal threshold, PWT) in Control and SCD mice. The von Frey test was conducted during the light phase of the day-night cycle. Each mouse was individually placed beneath an inverted plastic box with an elevated wire mesh bottom and was allowed to adjust to the environment for 1 hr. From below the mesh floor, a set of von Frey filament (Bioseb, Vitrolles, France) weights ranging from 0.04 g to 6 g were applied perpendicularly to the mid-plantar surface of the hind paw. The peak of force in grams was recorded. In each animal, triplicates of hind paw were taken with a 60-s interval between each stimulus. The test begins by assessing the response to 0.4 g filament. If there was no response, the next filament with a higher force was tested; if there was a response, the next lower force filament was tested. This continues until at least four readings were obtained after the first change of direction. Flinching, biting, or paw withdrawal during or immediately after applying the stimulus were considered nociceptive defensive behaviors and were noted as positive responses. Data was analyzed using the up and down method.

Thermal hyperalgesia: At 5 weeks after treatment with Vehicle or BGJ398, has commenced, the mice were brought into the testing room and acclimated for 1 h. For heat sensitivity use a hot plate (Ugo Basile) at 48-55 °C, was used. The mouse was placed on the testing apparatus and the timer was started. The paw withdrawal latency (PWL) to show a nociceptive response with hind paw lick, hind paw flick, or a jump was recorded. The mouse was immediately removed once this response was observed. If there was no response within 60 seconds, the test was terminated, and the mouse removed from the hot plate to prevent heat-related injury. Shorter PWL indicates increased heat sensitivity. The surface of the hot plate was cleaned with 70% ethanol between the testing of each mouse.

### Results

Six months old female healthy Control (Ctrl) and Sickle Cell Disease (SCD) were sq treated with vehicle or FGF Receptor signaling Inhibitor BGJ398, 5 days/week. Five weeks after treatment has commenced, pain behavior PWT by Von Frey testing was performed.

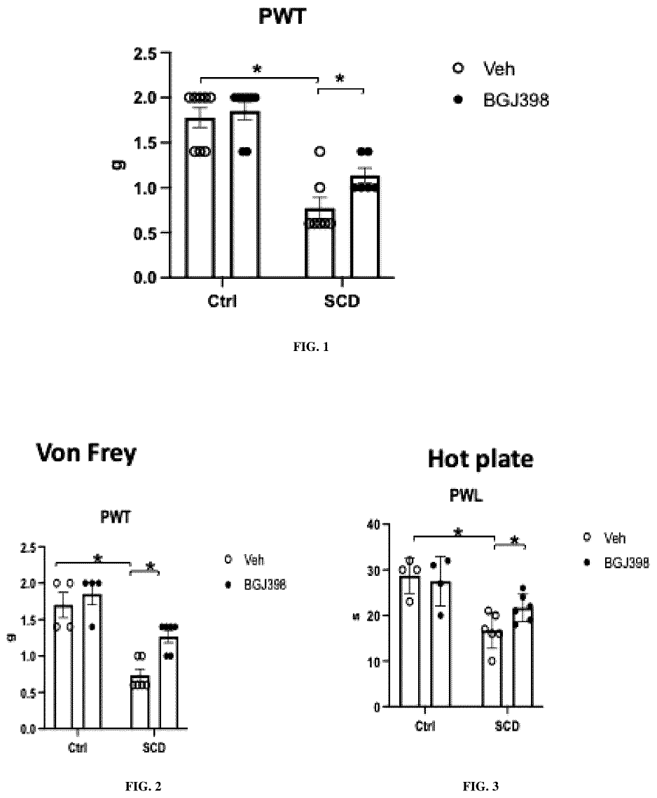

Significantly decreased PWT (decreased PWT indicates increased mechanical hyperalgesia) was significantly improved by BGJ398 (FIG. 1).

Eight months old male healthy Control (Ctrl) and Sickle Cell Disease (SCD) were sq treated with vehicle or FGF Receptor signaling Inhibitor BGJ398, 5 days/week. Five weeks after treatment commenced, mechanical hyperalgesia was tested by Von Frey analysis that showed the decreased PWT in SCD mice was significantly increased by BGJ398 (FIG. 2). Thermal hyperalgesia was tested by hot plate. Hot plate analysis showed that decreased PWL (decreased PWL indicates increased thermal hyperalgesia) in SCD was rescued by BGJ398 (FIG. 3).

### Example 2

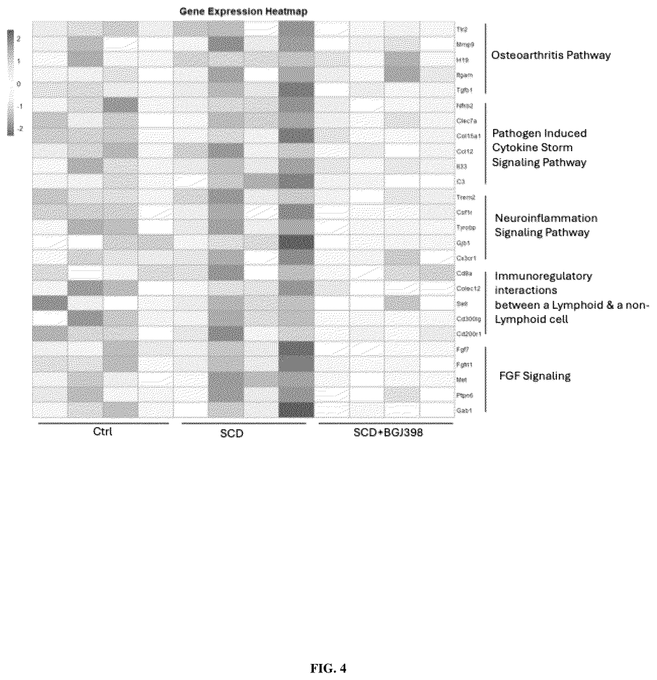

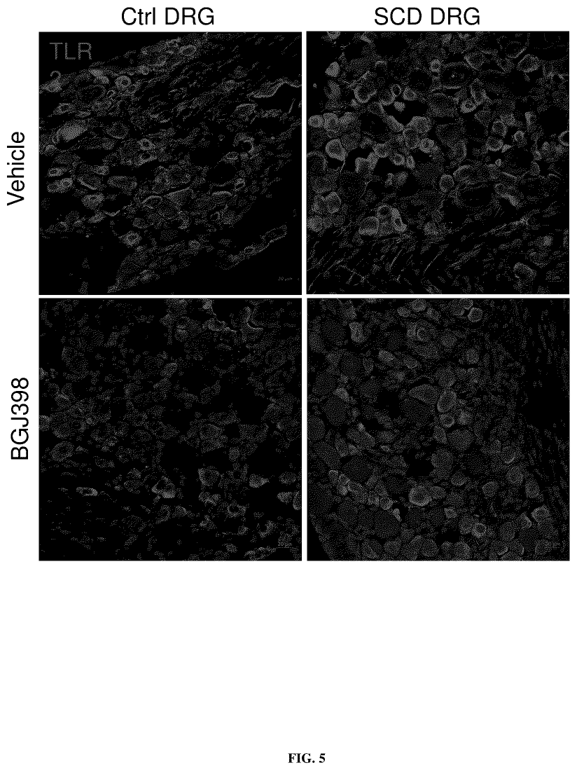

At 8 months of age, control (Ctrl) and SCD male mice received subcutaneous injections of vehicle or BGJ398 (2 mg/kg body weight/day), 5 days per week for 6 weeks. After 6 weeks, mice were sacrificed and L3-L5 dorsal root ganglia (DRG) were collected for RNA-seq analysis. The results from this experiment are provided in FIG. 4. As shown in this figure, certain genes in the osteoarthritis, pathogen induced cytokine storm signaling, immunoregulatory interactions between a lymphoid and non-lymphoid cell, and FGF signaling pathways were upregulated in DRG tissue from male SCD mice. However, following treatment with BGJ398, expression of these genes in the DRG decreased.

At 6 months of age, control (Ctrl) and SCD female mice received subcutaneous injections of vehicle or BGJ398 (2 mg/kg body weight/day), five days per week for six weeks. After 6 weeks, mice were sacrificed and L3-L5 dorsal root ganglia were collected for toll-like receptor 2 (TLR2) immunofluorescence (IF) analysis. The results from this experiment are provided in FIG. 5. As shown in this figure, TLR2, which is a key component of the innate immune system, was highly expressed in the DRGs of female SCD mice. However, following BGJ398 treatment, TLR2 expression was reduced in two out of three SCD samples.

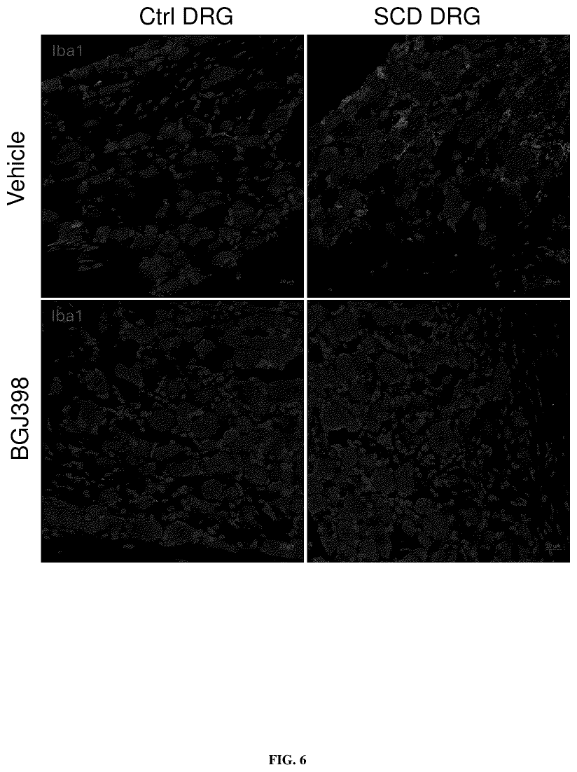

At 6 months of age, control (Ctrl) and SCD female mice received subcutaneous injections of vehicle or BGJ398 (2 mg/kg body weight/day), five days per week for six weeks. After 6 weeks, mice were sacrificed and L3-L5 dorsal root ganglia were collected for ionized calcium-binding adapter molecule 1 (IBA1) IF analysis. The results from this experiment are provided in FIG. 6. As shown in this figure, IBA1, which is a marker of microglia and macrophages, showed similar levels in vehicle- and BGJ398-treated control groups, but was visibly increased in the DRGs of female SCD mice. This increase was reduced in all SCD samples following BGJ398 treatment.

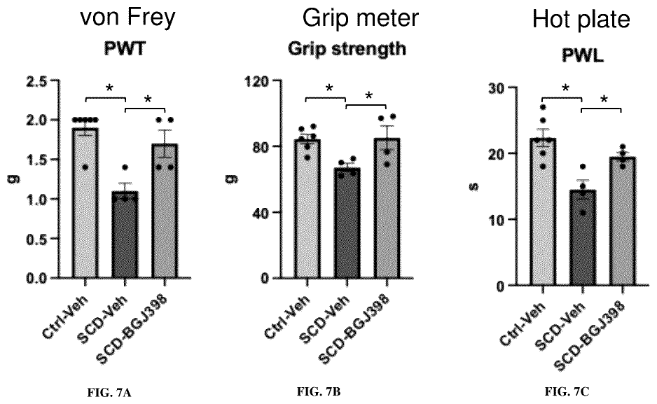

At 6 months of age, control (Ctrl) and SCD female mice received subcutaneous injections of vehicle or BGJ398 (1 mg/kg body weight/day), two days per week for six weeks. After 6 weeks, mice were assessed for mechanical hyperalgesia using von Frey analysis, deep tissue hyperalgesia using a grip strength meter, and thermal hyperalgesia using a hot plate assay. The results from this experiment are provided in FIGS. 7A-7C. As shown in FIG. 7A, a significant decrease in PWT, indicating increased mechanical hyperalgesia in SCD mice, was significantly improved by BGJ398 treatment. As shown in FIG. 7B, the reduced grip strength observed in SCD mice (reflecting increased deep tissue hyperalgesia) was significantly improved following BGJ398 treatment. As shown in FIG. 7C, decreased paw withdrawal latency (PWL), indicative of increased thermal hyperalgesia in SCD mice, was significantly rescued by BGJ398.

Chronic pain in both female and male SCD mice showed increased mechanical hyperalgesia and increased heat sensitivity, as compared to age- and sex-matched Ctrl mice, that was partially rescued by BGJ398 treatment. Moreover, BGJ398 treatment rescued increased deep tissue hyperalgesia in female SCD mice. Therefore, SCD mice show the characteristics of hyperalgesia reported in SCD patients, and BGJ398 has potential to ameliorate pain in SCD.

These results demonstrate that FGFR tyrosine kinase inhibition reduces multiple modalities of hyperalgesia in sickle cell disease by modulating dorsal root ganglion immune signaling, thereby supporting the claimed methods and compositions for treating SCD-associated pain.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. Any combination of the above-described elements in all possible variations thereof is encompassed by the disclosure unless otherwise indicated herein or otherwise clearly contradicted by context.

For reasons of completeness, various aspects of the disclosure are set out in the following numbered clauses:

Clause 1. A method of treating sickle cell disease-associated pain in a subject, comprising administering to the subject an effective amount of a fibroblast growth factor receptor (FGFR) inhibitor, wherein the administration reduces at least one of mechanical hyperalgesia, thermal hyperalgesia, or deep tissue hyperalgesia, thereby treating said pain.

Clause 2. The method of clause 1, wherein the FGFR inhibitor is a pan-FGFR inhibitor.

Clause 3. The method of clause 1, wherein the FGFR inhibitor is an FGFR tyrosine kinase inhibitor (FGFR-TKI).

Clause 4. The method of clause 3, wherein the FGFR-TKI inhibits FGFR1, FGFR2, and FGFR3.

Clause 5. The method of clause 1, wherein the FGFR inhibitor is selected from BGJ398/infigratinib, erdafitinib, pemigatinib, futibatinib, lirafugratinib, dovitinib, nintedanib, lenvatinib, and ponatinib, or a pharmaceutically acceptable salt thereof.

Clause 6. The method of clause 5, wherein the FGFR inhibitor is BGJ398 or a pharmaceutically acceptable salt thereof.

Clause 7. The method of clause 1, wherein the subject exhibits neuropathic and inflammatory pain components.

Clause 8. The method of clause 1, wherein the pain is associated with a vaso-occlusive episode.

Clause 9. The method of clause 1, wherein administration reduces mechanical hyperalgesia as measured by a von Frey assay.

Clause 10. The method of clause 1, wherein administration reduces thermal hyperalgesia as measured by a hot plate assay.

Clause 11. The method of clause 1, wherein administration improves grip strength indicative of reduced deep tissue hyperalgesia.

Clause 12. The method of clause 1, wherein the FGFR inhibitor is administered subcutaneously or orally.

Clause 13. The method of clause 1, wherein the FGFR inhibitor is administered chronically for at least four weeks.

Clause 14. The method of clause 1, wherein administration reduces pain without opioid administration.

Clause 15. The method of clause 1, wherein the subject is a human subject.

Clause 16. A method of modulating pain in a subject having sickle cell disease, comprising administering an FGFR tyrosine kinase inhibitor in an amount effective to reduce toll-like receptor 2 (TLR2) expression and/or ionized calcium-binding adapter molecule 1 (IBA1) expression in dorsal root ganglia relative compared to a vehicle control, thereby modulating pain.

Clause 17. The method of clause 16, wherein TLR2 expression is reduced by at least about 1.5-fold relative to a vehicle control.

Clause 18. The method of clause 16, wherein IBA1 expression is reduced by at least about 1.5-fold relative to a vehicle control.

Clause 19. The method of clause 16, wherein the FGFR tyrosine kinase inhibitor is BGJ398.

Clause 20. The method of clause 16, wherein reduced TLR2 and/or IBA1 expression correlates with reduced hyperalgesia.

Clause 21. The method of clause 16, wherein the reduction occurs in the lumbar dorsal root ganglia.

Clause 22. The method of clause 16, wherein pain modulation is achieved in the absence of opioid therapy.

Clause 23. A pharmaceutical composition for treating sickle cell disease-associated pain, comprising: (a) a fibroblast growth factor receptor (FGFR) inhibitor; and (b) a pharmaceutically acceptable carrier.

Clause 24. The composition of clause 23, wherein the FGFR inhibitor is BGJ398.

Clause 25. The composition of clause 23, formulated for subcutaneous administration.

Clause 26. The composition of clause 23, formulated for oral administration.

Clause 27. The composition of clause 23, wherein the composition is suitable for chronic administration.

Clause 28. The composition of clause 23, wherein the composition is provided in a kit with instructions for treating sickle cell disease-associated pain.

Clause 29. The composition of clause 23, for use in reducing mechanical and thermal hyperalgesia.

Clause 30. The composition of clause 23, for use in treating pain associated with vaso-occlusive episodes.

Clause 31. A method of treating pain associated with sickle cell disease in a subject in need thereof, comprising administering an effective amount of an FGFR inhibitor, wherein the FGFR inhibitor is BGJ398/infigratinib or a pharmaceutically acceptable salt thereof.

Clause 32. A method of reducing hyperalgesia in a subject in need thereof, the method comprising administering to the subject an effective amount of a fibroblast growth factor receptor (FGFR) inhibitor, thereby reducing hyperalgesia in the subject.

Clause 33. The method of clause 32, wherein the hyperalgesia is associated with sickle cell disease in the subject.

Clause 34. The method of clause 32, wherein the FGFR inhibitor is an FGFR tyrosine kinase inhibitor.

Clause 35. The method of clause 32, wherein the FGFR tyrosine kinase inhibitor is BGJ398, or a pharmaceutically acceptable salt, enantiomer, stereoisomer, solvate, polymorph, or prodrug thereof.

Clause 36. The method of clause 32, wherein the hyperalgesia is mechanical hyperalgesia.

Clause 37. The method of clause 32, wherein the hyperalgesia is deep tissue hyperalgesia.

Clause 38. The method of clause 32, wherein the hyperalgesia is thermal hyperalgesia.

Clause 40. The method of clause 32, wherein the reduction in hyperalgesia is measured by von Frey analysis, grip strength measurement, hot plate assay, or quantitative sensory testing.

### Incorporation By Reference

All U.S. and PCT patent publications and U.S. patents mentioned herein are hereby incorporated by reference in their entirety as if each individual patent publication or patent was specifically and individually indicated to be incorporated by reference. However, where such reference is made, and whether to patents, publications, non-patent literature, or other sources of information, it is for the general purpose of providing context for discussing features of the invention. Accordingly, unless specifically stated otherwise, the reference is not to be construed as an admission that the document or underlying information, in any jurisdiction, is prior art, or forms part of the common general knowledge in the art. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating pain in a subject, the method comprising administrating to the subject an effective amount of a fibroblast growth factor receptor (FGFR) inhibitor, thereby treating pain in the subject.

2. The method of claim 1, wherein the FGFR inhibitor is a pan-inhibitor.

3. The method of claim 1, wherein the FGFR inhibitor is an FGFR tyrosine kinase inhibitor (FGFR-TKI).

4. The method of claim 1, wherein the FGFR inhibitor is selected from the group consisting of BGJ398, erdafitinib, pemigatinib, futibatinib, lirafugratinib, dabogratinib, irpagratinib, nintedanib, lenvatinib, dovitinib, and ponatinib, or pharmaceutically acceptable salt, enantiomer, stereoisomer, solvate, polymorph, or prodrug thereof.

5. The method of claim 4, wherein the FGFR inhibitor is BGJ398 or pharmaceutically acceptable salt, enantiomer, stereoisomer, solvate, polymorph, or prodrug thereof.

6. The method of claim 1, wherein the FGFR inhibitor is bemarituzumab.

7. The method of claim 1, wherein the pain is selected from the group consisting of pain associated with a sickle cell disease, somatic pain, neuropathic pain, inflammatory pain, musculoskeletal pain, visceral pain, nociplastic pain, acute pain, and chronic pain.

8. The method of claim 7, wherein the pain is associated with a sickle cell disease.

9. The method of claim 1, wherein the subject exhibits one or more symptoms selected from the group consisting of mechanical hyperalgesia, deep tissue hyperalgesia, and thermal hyperalgesia.

10. The method of claim 9, wherein administration of the FGFR inhibitor results in a reduction, elimination, or stabilization of one or more symptoms selected from the group consisting of mechanical hyperalgesia, deep tissue hyperalgesia, and thermal hyperalgesia.

11. The method of claim 1, wherein administration of the FGFR inhibitor decreases toll-like receptor 2 (TLR2) expression in the dorsal root ganglion (DRG) of the subject.

12. The method of claim 11, wherein administration of the FGFR inhibitor decreases TLR2 expression in the DRG of the subject by at least 1.5-fold, at least 2-fold, at least 2.5-fold, at least 3-fold, at least 3.5-fold, at least 4-fold, at least 4.5-fold, or at least 5-fold compared to administration of a vehicle control.

13. The method of claim 1, wherein administration of the FGFR inhibitor decreases ionized calcium-binding adapter molecule 1 (IBA1) expression in the DRG of the subject.

14. The method of claim 13, wherein administration of the FGFR inhibitor decreases IBA1 expression in the DRG of the subject by at least 1.5-fold, at least 2-fold, at least 2.5-fold, at least 3-fold, at least 3.5-fold, at least 4-fold, at least 4.5-fold, or at least 5-fold compared to administration of a vehicle control.

15. The method of claim 1, wherein administration of the FGFR inhibitor results in an improvement in the subject's score of a clinical assessment tool, as compared to a score of the clinical assessment tool prior to administration of the FGFR inhibitor, wherein the clinical assessment tool is selected from the group consisting of Numeric Rating Scale (NRS), Visual Analogue Scale (VAS), Wong-Baker FACES Pain Rating Scale, Behavioral Pain Scale, Short Form Health Survey 36, McGill Pain Questionnaire (MPQ), pressure algometry, Von Fey test, and Quantitative sensory testing (QST).

16. The method of claim 1, wherein the subject is a human subject.
